(19) United States
(12) Patent Application Publication
(10) Pub. No.: US 2007/0207457 A1
(43) Pub. Date: Sep. 6, 2007

(54) Methods For Obtaining Aptamers Using Microarray

(76) Inventors: Ryoichi Asai, Tokyo (JP); Shinichiro Nishimura, Tokyo (JP); Katsutoshi Takahashi, Tokyo (JP)

Correspondence Address: Peter G Carroll;Medlen & Carroll, Suite 350, 101 Howard Street, San Francisco, CA, 94105, US

(21) Appl. No.: 10/551,156

(22) PCT Filed: Mar. 24, 2004
(86) PCT No.: PCT/JP04/04102

§ 371 Date: Jan. 29, 2007

(30) Foreign Application Priority Data
Mar. 28, 2003 (JP) 2003-089494

(51) Int. Cl. C12Q 1/68; G06F 19/00

(52) U.S. Cl. 435/6; 702/20

(57) ABSTRACT

A microarray originally developed for analyzing intracellular RNA expressions can be used to easily load polynucleotides onto a chip and to instantaneously determine polynucleotides (aptamers) capable of binding to a target molecule. Namely, the present inventors discovered that a microarray can be used to rapidly and efficiently obtain aptamers that bind to a target molecule.

### TECHNICAL FIELD

The present invention relates to the biotechnology field, and in particular to methods for obtaining biopolymers.

### BACKGROUND ART

DNAs and RNAs are molecules that are responsible for genetic information in organisms. Some single-stranded DNAs and RNAs comprise nucleotide sequences that bind specifically to target molecules. They are called aptamers, and can be obtained mainly by the SELEX method (see Ellington, A. D. and Szostak, J. W., Nature, Vol. 346, p. 818-822, 1990; Tuer, K. C. and Gold, L., Science, Vol. 249, p. 505-510, 1990). This is a method for searching DNAs or RNAs that function as aptamers by producing DNAs or RNAs having nucleotide sequences of a specific length at random, and then screening for DNAs or RNAs that bind to a target molecule. In this method, however, sequences need to be identified with a sequencer in the end because all the sequence populations are dissolved in one solution. Apart from this method, methods that search for functional polynucleotides (or polypeptides) by examining sequences one by one have been contemplated. According to these methods, amino acid sequences are individually determined to select sequences with a relatively high activity, from which novel sequences are generated by genetic algorithm or exon shuffling. These novel sequences are further determined individually, and then the processes are repeated many times to search for sequences with high activities (see International Publication WO 99/11818; and Yokobayashi, Y., J. Chem. Soc., Perkin Trans. Vol. 1, p. 2435-2437, 1996).

The SELEX method can be used to handle a very large population of sequences, but demands time, energy, and cost to determine all the sequences that are dissolved in one solution. On the contrary, according to the method by Yokobayashi et al., sequences are individually determined at first, however, synthesis and analysis of sequences demand time, energy, and cost for a large sequence population.

Regardless that methods of selecting primers for the differential display method (see Japanese Patent Application Kokai Publication No. (JP-A) 2000-308487 (unexamined, published Japanese patent application)) and biosensors equipped with aptamers (JP-A 2002-207026) have been known up to date, no methods that can be used for efficient aptamer selection within a short time and at a low cost are available.

### DISCLOSURE OF THE INVENTION

The present invention has been accomplished in view of these circumstances. An objective of the present invention is to provide methods for obtaining aptamers with a higher efficiency than conventional methods.

The present inventors dedicated themselves to research in order to solve the above-mentioned problems. The conventional method by Yokobayashi et al. is inadequate for processing many sequences because more time is needed to synthesize and analyze the sequences as they increase in number. The present inventors, on the other hand, successfully reduced the labor by employing microarrays. A microarray allows automatic synthesis of a specified sequence at a designated position on a chip, and large numbers of sequences as many as thousands can be arrayed. DNA microarray is generically classified into two types. One type of DNA microarray loads PCR-amplified products onto a chip, and the other type can synthesize oligonucleotides on a chip in series and load oligonucleotides that are approximately 30 bases onto a chip. Although originally developed to analyze expressions of intracellular RNAs, the latter microarray can be used to easily load oligonucleotides that have one or two base substitutions onto a chip. Thus, the present inventors realized that the system can also be used to search for aptamers. Further, the performances of many aptamers can be determined instantly and simultaneously by using a fluorescent label and an exclusive-use scanner. The present inventors discovered for the first time that a microarray can be used to rapidly and efficiently obtain target molecule-binding aptamers.

Namely, the present invention relates to methods for obtaining aptamers using microarrays, and more specifically, it provides:
[1] a method for obtaining an aptamer, comprising the following steps (a) to (e) with steps (b) to (e) repeated any number of times:

(a) immobilizing to a microarray substrate a plurality of polynucleotides comprising nucleotide sequences that are different from one another;

(b) contacting a labeled target molecule with said microarray substrate immobilized with polynucleotides;

(c) determining the binding strengths of said polynucleotides to said target molecule;

(d) selecting one or more polynucleotides having relatively high binding strengths; and

(e) immobilizing each of the polynucleotides selected by step (d) to a microassay substrate, wherein a mutation is introduced into said polynucleotide nucleotide sequences;
[2] the method of [1], wherein the mutation in step (e) is a one- or two-base substitution mutation;
[3] the method of [1] or [2], wherein the labeling is fluorescence labeling;
[4] the method of any one of [1] to [3], wherein the contact in step (b) is carried out by immersing the microarray substrate in a solution in which the target molecule has been dissolved; and
[5] the method of any one of [i] to [4], wherein the polynucleotides in step (a) comprise computer-generated random sequences.

The present invention relates to methods for obtaining aptamers characterized by the use of microarrays.

In a preferred embodiment of the present invention, a method obtains an aptamer capable of binding to a target molecule at a given position on a microarray using the signal strength as an index. Namely, the present invention is a method for obtaining an aptamer capable of binding to a target molecule, where the binding activity between the target molecule and a test polynucleotide (i.e., an aptamer candidate) immobilized on a microarray substrate (herein, sometimes simply referred to as “substrate”) is used as an index.

The “aptamer” of the present invention refers to a nucleic acid molecule (such as polynucleotide) capable of binding to a target molecule, and can be schematically demonstrated in FIG. 1, for example. Generally, the type and length of the nucleotide sequence can be changed so as to bind various target molecules. The “polynucleotide” of the present invention includes the so-called “oligonucleotides”.

Microarray is generally referred to as a device comprising a substrate immobilized with arrays of polynucleotides or such, wherein the surface of the substrate loaded with nucleotides is usually made of glass, silicon, or such. High-density arrays prepared by simultaneously synthesizing multiple types of polynucleotides on a substrate are also called DNA chips. The microarray of the present invention is not limited to the so-called “attachment-type” microarrays, and includes the so-called “chips” that have polynucleotides synthesized on a substrate.

The “substrate” in the present invention refers to a plate-like material that enables immobilization of nucleotides, and is not particularly limited as long as it can immobilize nucleotides. The substrates generally used in the microarray technology, for example, those made of glass or silicon, can be preferably used.

A microarray generally comprises thousands of polynucleotides spotted on a substrate at high densities. The process of immobilizing polynucleotides onto a substrate is also called “printing”. The polynucleotides are generally spotted (printed) on the surface of a non-porous substrate. The surface of a substrate is generally made of glass, and may be made of a porous membrane such as a nitrocellulose membrane. The polynucleotides can be synthesized in situ on a polynucleotide array. Methods of in situ oligonucleotide synthesis using, for example, the photolithographic technique (Affymetrix) and the inkjet technique (Rosetta Inpharmatics) for immobilizing chemicals are already known, and any of these techniques may be employed to prepare the substrates of the present invention. “Immobilization” onto the substrates of the present invention also includes the so-called “synthesis”. One skilled in the art can generally use a commercially available device that enables high-density spotting (printing) to prepare a suitable microarray in laboratory, for example, one that comprises ten thousands types of spots (prints) or more on a slide glass.

In the present invention, polynucleotides may be artificially synthesized and immobilized onto a substrate, wherein the polynucleotides may be synthesized by standard means known in the art, for example, employing a commercially available automatic DNA synthesizer.

In the present invention, a preferred embodiment of the method for obtaining an aptamer comprises steps (a) to (e) described below:

(a) immobilizing to a microarray substrate a plurality of polynucleotides comprising nucleotide sequences that are different from one another;

(b) contacting a labeled target molecule with said microarray substrate immobilized with polynucleotides;

(c) determining the binding strengths of said polynucleotides to said target molecule;

(d) selecting one or more polynucleotides having relatively high binding strengths; and

(e) immobilizing each of the polynucleotides selected by step (d) to a microassay substrate, wherein a mutation is introduced into said polynucleotide nucleotide sequences.

The target molecules in the present invention are not particularly limited, and include naturally occurring compounds, synthetic compounds, peptides, and non-peptide compounds. Further, cell extracts, cell culture supernatants, products of fermentation microorganisms, marine organism extracts, plant extracts, purified and crude proteins, and molecules isolated and purified therefrom, may also be used as a target molecule. More specifically, substances that are applicable as sensor elements or disease biomarkers and such may be used as a target molecule. Examples of sensor elements include hepatotoxin microcystine released from water pollutants, cyanobacteria and such, and α-fetoprotein which is a cancer indicator substance.

Preferably, the target molecules of the present invention are fluorescently labeled or are fluorescent molecules themselves.

One skilled in the art can fluorescently label a target molecule using known methods by considering the target molecule type. When the target molecule is a protein, it can be fluorescently labeled by suitably using, for example, an amino group labeling method or a sulfhydryl group (—SH) labeling method. In these methods, a fluorescent substance can be linked to a target molecule via the amino group of a lysine residue or the sulfhydryl group of a cysteine residue because they are generally comprised within or at the N terminus of a target molecule's protein/peptide/immune antibody sequence.

Fluorescence labeling of target molecules generally utilizes a fluorescence-labeling substance that absorbs and emits lights of discemable wavelengths. In the microarrays of the present invention, aptamer search for two or more target molecules in a single assay can be performed by using a plurality of fluorochromes having different colors.

Substances other than a fluorescent substance, such as a chemiluminescent substance or an electroactive substance, may be used for the labeling of the present invention.

The polynucleotides to be immobilized onto a substrate of the present invention are not limited to a particular type of sequence, and generally comprise random sequences. The “random sequences” can be suitably prepared by one skilled in the art using a computer. Since many aptamers have a stem structure, in one embodiment of the present invention, the above-mentioned polynucleotides can be designed to have several bases at both ends that are complementary sequences with each other to allow stem formation. However, such a design is not always necessary.

The length of the polynucleotides to be immobilized onto a substrate is not limited, and is generally 10 to 100 bases, preferably 20 to 80 bases, and more preferably 50 to 80 bases. Many reported that the target molecule-recognizing sites in aptamers are usually about 30 bases. Thus, it is preferable that the polynucleotides of the present invention have 30 bases or more.

In the present invention, generally a plurality of polynucleotides are immobilized to a substrate, and preferably have sequences that differ from each other. However, since identical sequences are considered possible among the plurality of random sequences obtained above, the polynucleotides to be immobilized to a substrate in the present invention are not limited to those that have sequences different from each other. The number of the types of polynucleotides to be immobilized to a substrate is not particularly limited, and is usually hundreds to tens of thousands. In the present invention, the number of the types of test polynucleotides to be immobilized to a substrate can be increased to attain a desirable aptamer efficiently.

For controls, a polynucleotide proven to be an aptamer to a target molecule (positive control) and a polynucleotide proven to be not an aptamer (negative control) may be preliminarily immobilized to a microarray substrate of the present invention. These controls are useful for selecting a polynucleotide that has high binding strength to the target molecule, or for determining whether a test polynucleotide is an aptamer or not.

For the microarray device in the present invention, one skilled in the art can use a commercially available microarray device, such as one developed by CombiMatrix Corporation and distributed by Roche Diagnostics.

The “contacting” in the above-described step (b) of the present invention is not particularly limited, and may be conducted, for example, under the following conditions.

A substrate of the present invention is immersed in 3×SSPE (20×SSPE; 0.2 M phosphate, pH 7.4 (±0.1, 25° C.), 2.98 M sodium chloride, 0.02 M EDTA) containing resorufin (Ex=571 nm, Em=585 nm) for incubation at 25° C. room temperature for 16 hrs.

Then, the substrate is preferably washed with the same solvent used for the above-mentioned “contacting”. It is preferable that the solvent to be used for washing does not contain a (fluorescent) labeling substance. Usually, the washing process is preferably repeated two or three times. The substrate can be washed to remove target molecules that did not bind to polynucleotides. In a preferred embodiment of the present invention, the substrate is then dried. For example, the washed substrate can be dried in a light-shielding dryer for about 1 hour.

In a preferred embodiment of the present invention, the binding strengths of a labeled target molecule to polypeptides immobilized on a substrate are determined. Polypeptides that fail to bind the target molecule do not produce signals, such as fluorescence, at their positions on the microarray substrate. A fluorescent label generally provides a greater fluorescence intensity (brightness of fluorescence) for a higher binding strength. The fluorescent signal on a microarray is generally detected by a fluorescence detector and usually can be detected using a known device, such as a confocal scanning device or a CCD (Charge Coupled Device) camera. Usually, a confocal scanner moves a substrate or a confocal lens in two dimensions to apply a laser beam to a microdomain on the substrate, and thereby excites the fluorescent molecule. The light emitted from the fluorescent sample at each position of the substrate is converted to electric signal data by a detector, such as a photo multiplier tube, and the data are then collected by a confocal scanner. A CCD camera also detects as a confocal scanner with the same principle. The fluorescence detector includes commercially available devices shown below:
Scanner type:
Scan Array 4000, 5000 (General Scanning), GMS418 Array Scanner (Takara Shuzo), etc:
CCD camera type:
Gene Tac 2000 (Genomic Solutions), etc.

Generally, a microarray as described above yields an enormous amount of data, and hence a computer installed with data analysis software is used to manage the correspondence between data and positions of polynucleotides immobilized to a substrate, as well as for data analysis.

When a target molecule is labeled using methods other than the fluorescence labeling method, chemiluminescence or electrochemistry, for example, can be used to determine its binding strength depending on the labeling method.

In the above-described step (d) of the present invention, an oligonucleotide that demonstrates the highest binding strength is preferably selected. However, the number of oligonucleotides to be selected is not limited to one, and a plurality of oligonucleotides that demonstrate high binding strengths may be selected (the sequence of an oligonucleotide thus selected is sometimes referred to as a “mother sequence”). To show one example, the top-ranking ten or 1% of the test oligonucleotides having a high binding strength may be selected as mother sequences.

In the present invention, the sequence of an oligonucleotide selected in step (d) described above is then used as a “mother sequence” to prepare a sequence (child sequence) that has mutations introduced into the mother sequence.

The type and number of the above-mentioned mutations is not particularly limited, and is preferably a substitution mutation of 1 to 10 bases, and more preferably a substitution mutation of 1 or 2 bases. The substitution mutation of multiple bases in one polynucleotide is not particularly limited, and is preferably a substitution mutation for multiple consecutive bases, and more preferably a substitution mutation for two neighboring bases. Further, in the present invention, the type of the above-mentioned mutation is not particularly limited to 'substitution mutation,” and includes other mutations such as “insertion mutation” and “deletion mutation”. Further, some of the multiple mother sequences can be crossed with each other to provide child sequences.

The above-mentioned “child sequence” can be suitably prepared by using a computer. There are not particular limitations to the type of child sequences to be prepared. Generally, it is preferable that as many types of child sequences are prepared.

In the present invention, the polynucleotides (child sequences) consisted of sequences having introduced mutations in step (d) described above are then immobilized to a microarray substrate. The polynucleotides comprising the introduced mutations mentioned above can be immobilized onto a substrate by the method described above.

A preferred embodiment of the present invention is a method that comprises repeating the above-described steps (b) to (e) any number of times following the step (e) described above.

The above steps are repeated to yield oligonucleotides having higher binding strengths to a target molecule. The oligonucleotides obtained at the end have the highest binding activity to the target molecule, and are hence considered to be the aptamers.

The number of repetitions described above is generally about 5 to 6, and is not particularly limited as long as the aptamers can be obtained. There are preferably as many repetitions as possible. When a polynucleotide that has been identified to be an aptamer is immobilized to a substrate of the present invention as a control, the processes are preferably repeated until the polynucleotide thus obtained has a binding strength to the target molecule equivalent to that of the control polypeptide.

Further, in the present invention, polypeptides may be used instead of test polynucleotides. It is possible for one skilled in the art to immobilize polypeptides onto a substrate.

### BRIEF DESCRIPTION OF THE DRAWINGS

FIG. 1 is a schematic diagram of an aptamer binding to a target molecule. The black circle in the center shows the target molecule. The sequence of the aptamer is shown in SEQ ID NO: 1.

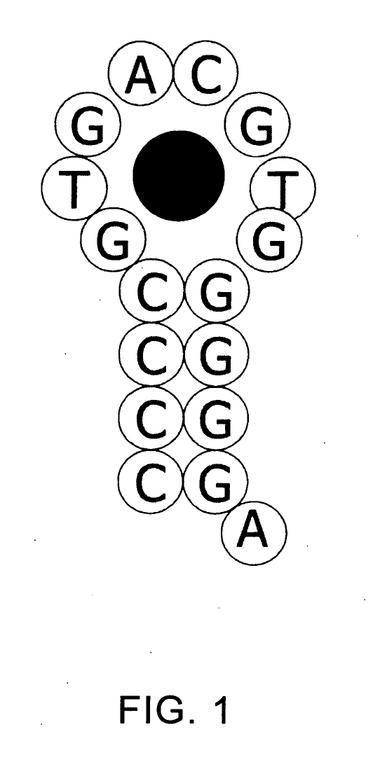

FIG. 2 is a diagram showing the standard procedure for one embodiment of the present invention.

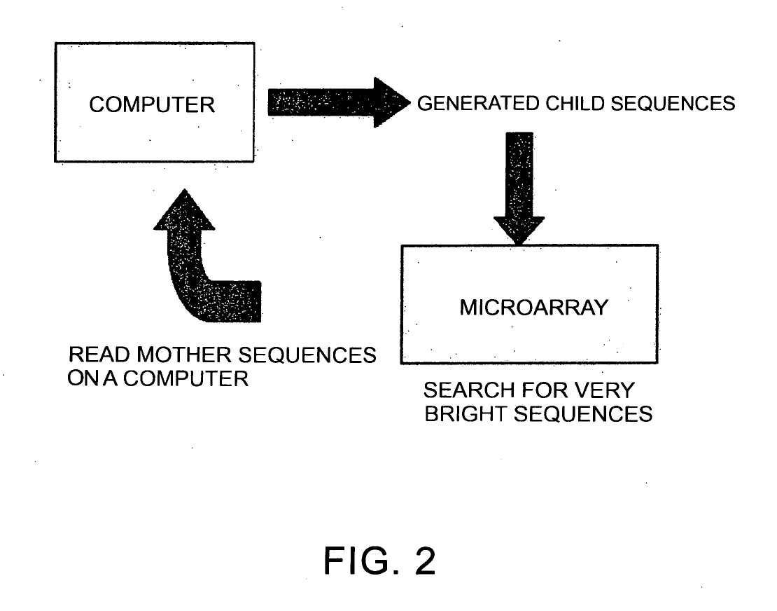

FIG. 3 is a photograph showing the fluorescence of a DNA chip, a chip where a specified DNA sequence was synthesized at each spot. A is a 0th generation chip aligned with random sequences. The mother sequence at the brightest spot was used to produce child sequences, and thereby formed B which is a 1st generation chip. Many spots were more fluorescent than those in the 0th generation. Furthermore, the child sequences at the brightest spots among them was used to form C which is a 2nd generation chip. Although none of the sequences on this chip were brighter than the child sequences, the chip had more fluorescent spots than the 1st generation chip.

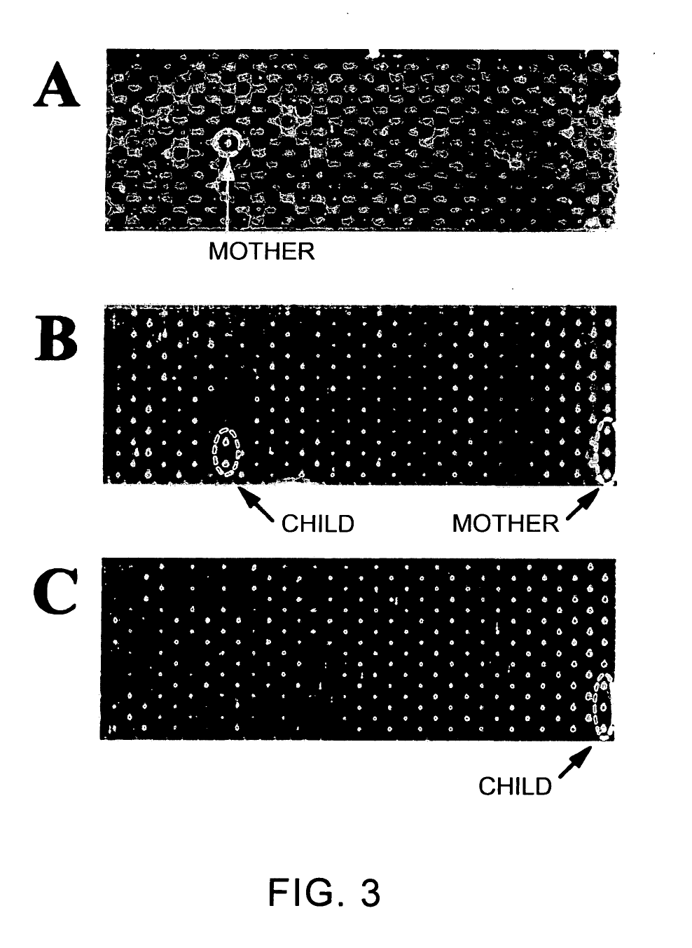

FIG. 4 is a diagram showing the fold result of a predicted secondary structure of the final sequence obtained. The sequence of the secondary structure is shown in SEQ ID NO: 2.

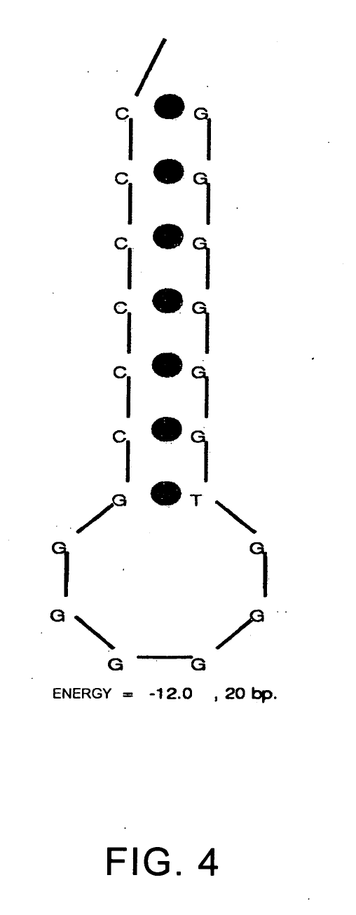

### BEST MODE FOR CARRYING OUT THE INVENTION

The present invention will be described in more detail with reference to the Example below, but is not limited thereto.

### EXAMPLE

### Aptamer Search for Resorufin—a Fluorochrome

Search was carried out for a polynucleotide (aptamer) that binds to resorufin—a fluorochrome. First, 20 base-polynucleotide sequences were prepared at random, provided that each sequence had C's for all five bases at the 5′ terminal end and G's for all five bases at the 3′ terminal side, thereby to allow stem formation via C-G bonds. The remaining 10 bases were selected at random, and a computer was used to prepare 300 different sequences. A chip for the exclusive use on a COMBIMATRIX-manufactured DNA synthesizer was readied for use, and specified sequences were synthesized at their respective spots on the chip. After the synthesis ended, the chip was immersed in a solution containing dissolved resorufin for a given time, and taken out, washed twice with a resorufin-free solvent, and dried. This chip was photographed using an Array WoRx-manufactured scanner to determine the fluorescence intensity. The sequence with the maximum intensity was specified, and used as a mother sequence to prepare child sequences on a computer according to the following methods.
(a) One-base substitution: only one of the 20 bases was substituted with a different base.
(b) Two-base substitution: two of the 20 bases were substituted, provided that they were next to each other.

The DNA synthesizer was again supplied with data from the child sequences prepared by the steps of (a) and (b), thereby to produce a DNA chip, which was then photographed by the scanner to determine the sequence which has a maximum intensity. The steps were repeated several times, finally to yield a polynucleotide with high strength, that is, an aptamer.

### INDUSTRIAL APPLICABILITY

The present invention provides methods for obtaining aptamers by using microarrays. The present invention is advantageous in that: (1) it does not require the use of PCR; (2) it is simple because the affinity strength can be determined by an on-chip binding test; (3) it allows a selection that is more mathematical than the SELEX method; (4) it does not require the reading of a DNA sequencer; and so on. Further, the methods of the present invention allow aptamers to be obtained with a DNA synthesizer and a scanner without special technical knowledge. If the price of blank chips can be reduced in the future, aptamer search can be done at very low cost.

Aptamers obtained by the methods of the present invention can be used in various applications. For example, the aptamers can be used as test reagents to determine pollutants in Kasumigaura or as therapeutic agents if they were to be developed into aptamers that inhibit virus proteins.

1. A method for obtaining an aptamer, comprising the following steps (a) to (e) with steps (b) to (e) repeated any number of times:
(a) immobilizing to a microarray substrate a plurality of polynucleotides comprising nucleotide sequences that are different from one another;
(b) contacting a labeled target molecule with said microarray substrate comprising immobilized polynucleotides;
(c) determining the binding strengths of said polynucleotides to said target molecule;
(d) selecting one or more polynucleotides having relatively high binding strengths; and
(e) immobilizing each of the polynucleotides selected by step (d) to a microassay substrate, wherein a mutation is introduced into said nucleotide sequences.

2. The method of claim 1, wherein the mutation in step (e) is a one- or two-base substitution mutation.

3. The method of claim 1, wherein the label is a fluorescent label.

4. The method of claim 1, wherein the contacting in step (b) is carried out by immersing the microarray substrate in a solution in which the target molecule has been dissolved.

5. The method of claim 1, wherein the polynucleotides in step (a) comprise computer-generated random sequences.

6. The method of claim 2, wherein the label is a fluorescent label.

7. The method of claim 2, wherein the contacting in step (b) is carried out by immersing the microarray substrate in a solution in which the target molecule has been dissolved.

8. The method of claim 3, wherein the contacting in step (b) is carried out by immersing the microarray substrate in a solution in which the target molecule has been dissolved.

9. The method of claim 2, wherein the polynucleotides in step (a) comprise computer-generated random sequences.

10. The method of claim 3, wherein the polynucleotides in step (a) comprise computer-generated random sequences.

11. The method of claim 4, wherein the polynucleotides in step (a) comprise computer-generated random sequences.


SEQUENCE LISTING (2 sequences)

SEQ ID NO: 1 - DNA, 17 bp, Artificial
  An artificially synthesized aptamer sequence
ccccgtgacgtggggga

SEQ ID NO: 2 - DNA, 20 bp, Artificial
  An artificially synthesized aptamer sequence
ccccccgggggggtgggggg